(19) United States
(12) Patent Application Publication
(10) Pub. No.: US 2021/0009649 A1
(43) Pub. Date: Jan. 14, 2021

(54) INTRON FOR INCREASING EXPRESSION LEVEL OF RHNGF

(71) Applicant: Xintrum Pharmaceuticals, Ltd., Nanjing (CN)

(72) Inventors: Hai Chen, Nanjing (CN); Hongliang Sun, Nanjing (CN); Yi Zhang, Nanjing (CN); Yuesheng Wang, Nanjing (CN)

(21) Appl. No.: 17/030,319

(22) Filed: Sep. 23, 2020

Related U.S. Application Data
(63) Continuation of application No. PCT/CN2018/114605, filed on Nov. 8, 2018.

(30) Foreign Application Priority Data
Mar. 26, 2018 (CN) 201810251703.X

(51) Int. Cl. C07K 14/48; C12N 15/85

(52) U.S. Cl. CPC C07K 14/48; C12N 2830/42; C12N 15/85

(57) ABSTRACT

A gene combination for expressing recombinant human nerve growth factor (rhNGF) includes an rhNGF precursor gene and an intron. The intron can have a nucleotide sequence shown in SEQ ID NO: 1 or SEQ ID NO: 2. A eukaryotic expression vector for rHNGF expression including the gene combination, a CHO cell including the eukaryotic expression vector, and methods for preparing rhNGF using the gene combination, the expression vector, and the CHO cell are also disclosed.

### TECHNICAL FIELD

The present invention relates to the DNA sequence of an intron that can increase the expression level of recombinant human nerve growth factor (rhNGF). The present invention also relates to the use of the intron in rhNGF preparation.

### DESCRIPTION OF RELATED ART

rhNGF is synthesized in vivo in the form of a precursor (proNGF), which includes a signal peptide, a pro-peptide, and a mature-rhNGF moiety. The signal peptide contributes to the secretion of proteins. The pro-peptide has two partially conserved regions that are required for proNGF expression, the formation of bioactive proteins by enzymatic hydrolysis, and the secretion of mature NGF, and that also contribute to the correct folding of proteins. proNGF has a potential N-glycosylation site, and glycosylation of the pro-peptide of proNGF helps the precursor exit from the endoplasmic reticulum. proNGF forms bioactive mature NGF after being hydrolyzed at particular sites with furin or prohormone convertase. Mature NGF has 118 amino acids and forms a two-chain dimer structure in which each chain has six cysteine residues capable of forming three pairs of intrachain disulfide bonds (Cys⁵⁸-Cys¹⁰⁸, Cys⁶⁸-Cys¹¹⁰, and Cys¹⁵-Cys⁸⁰). Proper formation of the disulfide bonds is essential to the activity of NGF.

One technical problem that needs to be solved now is how to express rhNGF efficiently in a eukaryotic expression system. An efficient expression vector is a major factor in achieving a high yield of rhNGF. Using a proper expression regulation sequence and a reasonable structural arrangement in the construction of an rhNGF expression vector can increase the expression level of rhNGF. In addition, the stability and transport efficiency of mRNA can be enhanced by the splicing out of introns. Therefore, the expression level of a target protein can be increased by using a suitable intron.

### SUMMARY OF THE INVENTION

One objective of the present invention is to obtain a suitable expression regulation sequence that can be used to construct an efficient expression vector so as to achieve a high yield of rhNGF.

An intron gene capable of increasing the expression level of rhNGF was discovered for the first time by the inventors of the present invention. The gene can increase the expression level of rhNGF significantly in a eukaryotic expression system.

The intron has the nucleotide sequence shown in SEQ ID NO. 1 (hereinafter referred to as glo for short) or SEQ ID NO. 2 (hereinafter referred to as aden for short).

Experiments with gene combinations containing the intron of the present invention in addition to a signal peptide and proNGF have proved that the intron can greatly increase the expression level of rhNGF.

The gene of the proNGF has the nucleotide sequence shown in SEQ ID NO. 7 and includes a pro-peptide and a mature-hNGF moiety. The amino acid sequence coded by the sequence of SEQ ID NO. 7 is shown in SEQ ID NO. 8.

Each gene combination is constructed into an expression vector.

The expression vectors are eukaryotic expression vectors and can be introduced into host cells by transient transfection or stable transfection.

The host cells are mammalian cells. The mammalian cells are Chinese hamster ovary (CHO) cells, human embryonic kidney 293 cells, COS cells, or Hela cells.

More specifically, the inventors of the present invention conducted the following research work:

1. The inventors searched for the amino acid sequences of hNGF in the protein sequence database UniProtKB and obtained the proNGF sequence of ID No. P01138, as shown in SEQ ID NO. 8. The proNGF amino acid sequence was reverse-translated by GenScript Biotech Corporation as per the features of CHO cell expression, and a DNA sequence was synthesized accordingly by GenScript Biotech Corporation as shown in SEQ ID NO. 7.

2. The 5′ end of the proNGF was added separately with the signal peptides Pre and Luc (whose nucleotide sequences are shown in SEQ ID NO. 3 and SEQ ID NO. 5 respectively) to obtain different signal peptide-proNGF gene combinations. Each gene combination was inserted into a eukaryotic expression vector, which in turn was introduced into a CHO cell by transient transfection. After culturing, a supernatant was obtained by centrifugation, and the rhNGF content of the supernatant was determined by ELISA. Then, using the natural signal peptide of the proNGF as a reference (the gene sequence of the natural signal peptide is shown in SEQ ID NO. 9 and is hereinafter referred to as Nat for short, and the amino acid sequence coded by the gene sequence is shown in SEQ ID NO. 10), the rhNGF expression levels respectively induced by the different signal peptides were compared.

3. The introns glo (as shown in SEQ ID NO. 1) and aden (as shown in SEQ ID NO. 2) were constructed separately into the expression vectors in 2 by means of a restriction endonuclease to obtain eukaryotic expression vectors containing an intron-signal peptide-proNGF gene combination. Then, using the vectors in 2 as a reference, the influence of the introns on the transient expression of rhNGF was assessed by the method mentioned in 2.

4. The expression vectors in 3 were transfected separately into CHO cells. Puromycin and methotrexate (MTX) were also added into the cells, and two rounds of pressurization screening were carried out to obtain cell pools.

5. Cell pools with high specific yields and good cell growth were selected for monocloning by the limiting dilution method. The resulting monoclones were screened to obtain an engineered cell strain that expressed rhNGF efficiently.

6. The growth curve, cell viability, and rhNGF expression level variation trend of the engineered cell strain, which was cultured in a bioreactor, were determined.

7. The biological activity of the rhNGF was assayed by TF-1 cell/MTS colorimetry.

Experiments have shown that the introns provided by the present invention can substantially increase the transient expression level of rhNGF (see embodiment 1), and that the supernatant of the engineered cell strain, which was constructed with one of the aforesaid intron-signal peptide-proNGF gene combinations and was cultured in a bioreactor, had an rhNGF expression level as high as 78 mg/L (see embodiments 2 and 3), with the biological activity of the rhNGF in the supernatant being equivalent to that of the international standard and higher than that of an mNGF for injection use (see embodiment 4).

### BRIEF DESCRIPTION OF THE SEVERAL VIEWS OF THE DRAWINGS

FIG. 1: The two plots respectively show two experiment results regarding the influence of a “signal peptide-proNGF” gene combination (which contains no intron) on the transient expression of rhNGF.

FIG. 2: The two plots respectively show two experiment results regarding the influence of an “intron-signal peptide-proNGF” gene combination on the transient expression of rhNGF.

FIG. 3: Schematic drawing of a eukaryotic expression vector for rhNGF, wherein the vector contains an “intron-signal peptide-proNGF” gene combination.

The intron, the signal peptide, the proNGF, and the mature-NGF moiety sequence (rhNGF) constitute a complete recombinant gene combination.

FIG. 4: SDS-PAGE analysis results of the rhNGF in the supernatants of the batch cultures of six cell strains, wherein all the rhNGF was purified with Capto-S before the analysis.

FIG. 5: Growth curves, cell viabilities, and rhNGF expression level variation trends of the engineered cell strain cultured in a bioreactor.

FIG. 6: Curves representing the biological activities of various NGF samples in inducing the proliferation of TF-1 cells, as determined by TF-1 cell/MTS colorimetry.

### SEQUENCE LISTING

SEQ ID NO. 1: Nucleotide sequence of the intron glo.
SEQ ID NO. 2: Nucleotide sequence of the intron aden.
SEQ ID NO. 3: Nucleotide sequence of the signal peptide Pre.
SEQ ID NO. 4: Amino acid sequence of the signal peptide Pre.
SEQ ID NO. 5: Nucleotide sequence of the signal peptide Luc.
SEQ ID NO. 6: Amino acid sequence of the signal peptide Luc.
SEQ ID NO. 7: Nucleotide sequence of a proNGF.
SEQ ID NO. 8: Amino acid sequence of the proNGF.
SEQ ID NO. 9: Nucleotide sequence of the signal peptide Nat.
SEQ ID NO. 10: Amino acid sequence of the signal peptide Nat.

### DETAILED DESCRIPTION OF THE INVENTION

The following embodiments serve only to demonstrate the method and apparatus of the present invention and are not intended to be restrictive of the scope of the present invention.

Embodiment 1: Creation of the Gene Combinations for Expressing rhNGF
1. Obtainment of the proNGF Gene

A search for the amino acid sequences of rhNGF was made in the protein sequence database UniProtKB, and the sequence of ID NO. P01138 was obtained. The amino acid sequence of proNGF consists of a pro-peptide with 103 amino acids and a mature-hNGF moiety with 120 amino acids, in which the two amino acids (RA) at the C terminal have no impact on the biological activity of the NGF and do not exist in natural NGF proteins, either. Therefore, the proNGF amino acid sequence used in the present invention dispensed with two amino acids and consisted of the 103-amino-acid pro-peptide and a 118-amino-acid hNGF moiety, as shown in SEQ ID NO. 8. The amino acid sequence was optimized and reverse-translated by GenScript Biotech Corporation in accordance with the features of CHO cell expression, and a DNA sequence was synthesized accordingly by GenScript Biotech Corporation as shown in SEQ ID NO. 7.

### 2. Selection of the Expression System

A mammalian cell-based expression system was chosen for expressing rhNGF. The expression system included CHO cells and eukaryotic expression vectors. The CHO cells served as host cells. Each eukaryotic expression vector had two insertion sites and was capable of expressing two genes simultaneously, or the second expression unit might be removed as needed in order to construct a monogenic expression vector. Each vector also included a dihydrofolate reductase selection label and a puromycin-resistant gene and was subjectable to MTX and puromycin pressurization screening at the same time for higher screening quality.

### 3. Screening of the Signal Peptide Elements

The signal peptides Pre and Luc were chosen to induce secretory expression of rhNGF, and were compared with the natural signal peptide Nat of rhNGF.

3.1 Obtainment of the Signal Peptide-proNGF Gene Combinations

The signal peptide Pre (whose gene sequence is shown in SEQ ID NO. 3) and the proNGF gene sequence were assembled by GenScript Biotech Corporation to obtain a sequence hereinafter referred to as Pre-pro-rhNGF for short. The 5′ end and the 3′ end of the sequence were added with an AvrII restriction site and a BstZ17I restriction site respectively, and the resulting sequence was constructed into a pUC57 plasmid to form a plasmid hereinafter referred to as pUC57-Pre-pro-rhNGF. The gene Pre-pro-rhNGF was obtained by double digestion of the plasmid pUC57-Pre-pro-rhNGF with the restriction endonucleases AvrII and BstZ17I. The Pre-pro-rhNGF gene segment obtained through double digestion was about 770 bp in size and was purified with a gel extraction kit for later use. Using pUC57-Pre-pro-rhNGF as a template, the signal peptides Luc and Nat (whose gene sequences are shown in SEQ ID NO.5 and SEQ ID NO. 9 respectively) were separately added through a primer to the 5′ end of the proNGF gene by PCR to obtain two gene segments of about 740 bp, which are hereinafter respectively referred to as Luc-pro-rhNGF and Nat-pro-rhNGF for short. The PCR products were purified with a common DNA purification kit and were subjected to UV spectrophotometry in order to determine their concentrations. The purified genes Luc-pro-rhNGF and Nat-pro-rhNGF were double-digested with the restriction endonucleases AvrII and BstZ17I and then purified for later use.

3.2 Construction of Eukaryotic Expression Vectors Containing the Signal Peptide-proNGF Gene Combinations

In order for each proNGF gene containing a different signal peptide to be constructed into a eukaryotic expression vector at a site downstream of the EF2/CMV hybrid promoter, the expression vectors were double-digested with the restriction endonucleases AvrII and BstZ17I, and the digested products were purified with a DNA purification kit.

The AvrII and BstZ17I double-digested genes Pre-pro-rhNGF, Luc-pro-rhNGF, and Nat-pro-rhNGF were respectively ligated by a T4 DNA ligase to expression vectors that had been subjected to the same double digestion, and the ligated products were chemically transformed into TOP10 competent cells by a connexon. Monocolonies grown from the transformation were screened by colony PCR in order to find positive clones. The PCR product of an expression vector to which a target gene was successfully ligated was about 1000 bp in size, whereas the PCR product of an empty vector was about 260 bp in size.

PCR-screened positive clones p-Pre-pro-rhNGF-8, p-Nat-pro-rhNGF-1, and p-Luc-pro-rhNGF-5 were selected for sequencing, and a comparison of the resulting sequences shows that the signal peptide-proNGF gene combinations of the three vectors were consistent with their respective theoretical sequences.

To express rhNGF, the second expression unit (CMV/EF1 expression cassette) of the eukaryotic expression vectors was deleted while the first expression unit remained, leaving the vectors with only one expression unit. The CMV/EF1 expression cassette was removed by cleaving the vectors p-Pre-pro-rhNGF-8, p-Nat-pro-rhNGF-1, and p-Luc-pro-rhNGF-5, whose sequences had been determined to be correct, with the restriction endonuclease Sfil, and the cleaved products were purified with a DNA purification kit. A T4 DNA ligase was then used to allow self-ligation of the Sfil-cleaved vectors, and the ligated vectors were chemically transformed into TOP10 competent cells by a connexon. Monocolonies grown from the transformation were screened by colony PCR in order to find positive clones. The PCR product of a vector whose CMV/EF1 expression cassette was successfully removed showed no bands, whereas the PCR product of a vector whose CMV/EF1 expression cassette was not successfully removed had a band size of 535 bp.

Based on the colony PCR screening results, monocolonial extracted plasmids p-Pre-pro-rhNGF(SfiI)-1, p-Nat-pro-rhNGF(SfiI)-1, and p-Luc-pro-rhNGF(SfiI)-1, whose PCR products showed no bands, were selected for tests, namely by single digestion with SfiI and double digestion with AvrII and BstZ17I, conducted separately. No bands were cut off from the positive clones when single digestion with SfiI was performed, and bands about 740 bp in size were cut off when double digestion with AvrII and BstZ17I was performed.

The plasmids p-Pre/Nat/Luc-pro-rhNGF(SfiI)-1, whose sequences had been determined to be correct by enzyme digestion, were sequenced, and a comparison of the resulting sequences shows that the signal peptide-proNGF gene combinations inserted respectively into the three expression vectors were consistent with their respective design sequences.

3.3 Influence of the Signal Peptide-proNGF Gene Combinations on rhNGF Expression

The efficiency with which each signal peptide-proNGF gene combination expressed rhNGF was determined by transient transfection.

(1) Culture Conditions of CHO Cells

Culture medium FortiCHO was added with 8 mM glutamine to make the complete culture medium. The culture conditions are as follows: an orbital shaker with an orbit diameter of 2.5 cm and a rotation speed of 130 rpm, carbon dioxide concentration: 8%, and temperature: 37° C. The CHO cells were subcultured when their density reached 1.5˜2.5×10⁶/mL. The density after subculturing was 3˜5×10⁵/mL.

(2) Cell Transfection

Cell subculturing was carried out one day before transfection such that cell density became 5˜6×10⁵/mL. Cell density was adjusted to 1×10⁶/mL with the complete culture medium prior to transfection. An appropriate transfection volume was selected according to the objective of each experiment. In a 1.5-mL microcentrifuge tube, the linearized expression vector p-Pre/Nat/Luc-pro-rhNGF(SfiI)-1 was added in a proportion of 1.67 μg per 10⁶ transfection target cells, followed by optiPRO SFM such that the final volume was 50 μL per 10⁶ transfection target cells. The mixture was gently stirred until thoroughly mixed. In another 1.5-mL microcentrifuge tube, two reagents, namely the transfection reagent FreeStyle Max and optiPRO SFM, were added at 1.67 μL and 48.33 μL per 10⁶ cells respectively, and the mixture was gently stirred until thoroughly mixed. The diluted Max solution and the DNA solution were mixed at once and placed at room temperature for 10 min, or 20 min at most. The DNA:MAX mixed solution was then added by drops into the cell suspension, and the resulting mixture was immediately placed on the orbital shaker for incubation.

(3) Determination of rhNGF Expression Levels

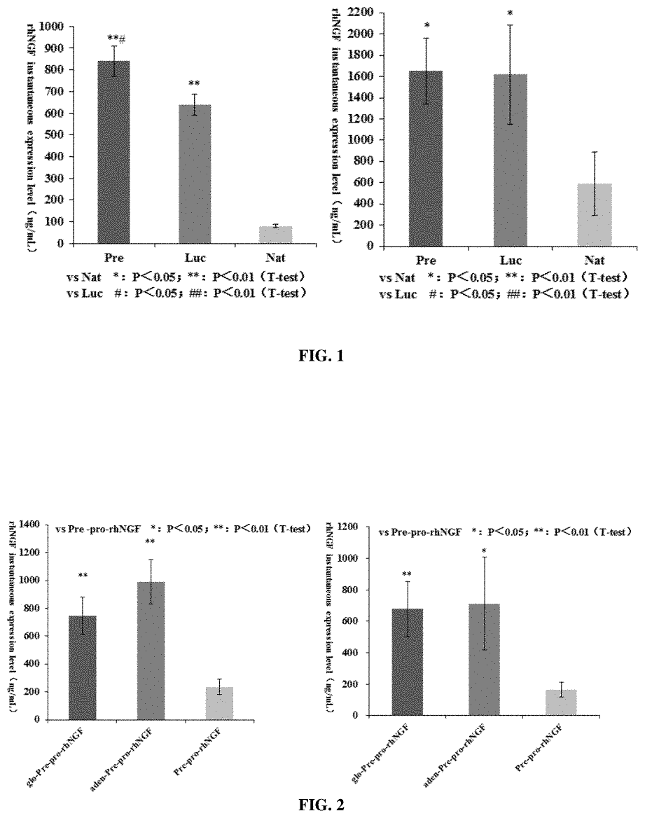

After transient transfection, the cells were cultured for 48 hours and then sampled. The rhNGF expression levels respectively induced by the different signal peptides were measured by ELISA, and each independent experiment was conducted twice as shown in FIG. 1. The experiment results show that the rhNGF expression levels corresponding to the signal peptide Pre/Luc-proNGF gene combinations were significantly higher than that corresponding to the natural NGF gene combination Nat-proNGF. The gene combinations containing the signal peptide Pre/Luc and proNGF were therefore the better choices for rhNGF expression.

### 4. Selection of the Intron Elements

The addition of an intron to the 5′ end of a target gene increases the stability of mRNA and thereby enhances the expression of the target protein. The introns glo and aden (whose sequences are shown in SEQ ID NO. 1 and SEQ ID NO. 2 respectively) were selected to be combined with the signal peptide and the proNGF gene, and the influence of these introns on rhNGF expression was investigated.

4.1 Obtainment of Introns

The DNA sequences of the introns glo and aden were synthesized by GenScript Biotech Corporation, were constructed in the same pUC57 plasmid, and were 150 bp and 296 bp in size respectively. Each sequence had an AvrII restriction site at each of its two ends, and the corresponding plasmid is hereinafter referred to as pUC57-glo-aden. glo and aden were obtained by single digestion of the plasmid pUC57-glo-aden with the restriction endonuclease AvrII, and the introns obtained were purified with a gel extraction kit for later use.

4.2 Construction of Eukaryotic Expression Vectors Containing Intron-Signal Peptide-proNGF Gene Combinations

The vector p-Pre-pro-rhNGF(SfiI)-1, which had a relatively high transient expression level of rhNGF in 3, was selected for the addition of introns. The vector was cleaved with the restriction endonuclease AvrII, and the cleaved product was purified with a common DNA purification kit. The AvrII-cleaved intron segments glo and aden were each ligated to a vector p-Pre-pro-rhNGF(SfiI) by a T4 DNA ligase, and the ligated products were chemically transformed into TOP10 competent cells by a connexon. Monocolonies grown from the transformation were screened by colony PCR in order to find positive clones, and the positive clones found were named p-glo-Pre-pro-rhNGF(SfiI) and p-aden-Pre-pro-rhNGF(SfiI) respectively. The gene combinations in these two vectors were sequenced, and a comparison of the resulting sequences shows that the sequences of the introns glo and aden were consistent with their respective design sequences. Thus, eukaryotic expression vectors containing the aforesaid intron-signal peptide-proNGF gene combinations were obtained.

4.3 Influence of the Intron-Signal Peptide-proNGF Gene Combinations on rhNGF Expression

The efficiency with which each intron-signal peptide-proNGF gene combination expressed rhNGF was also determined by transient transfection. The CHO cell culture conditions and the cell transfection method were the same as stated in 3.3.

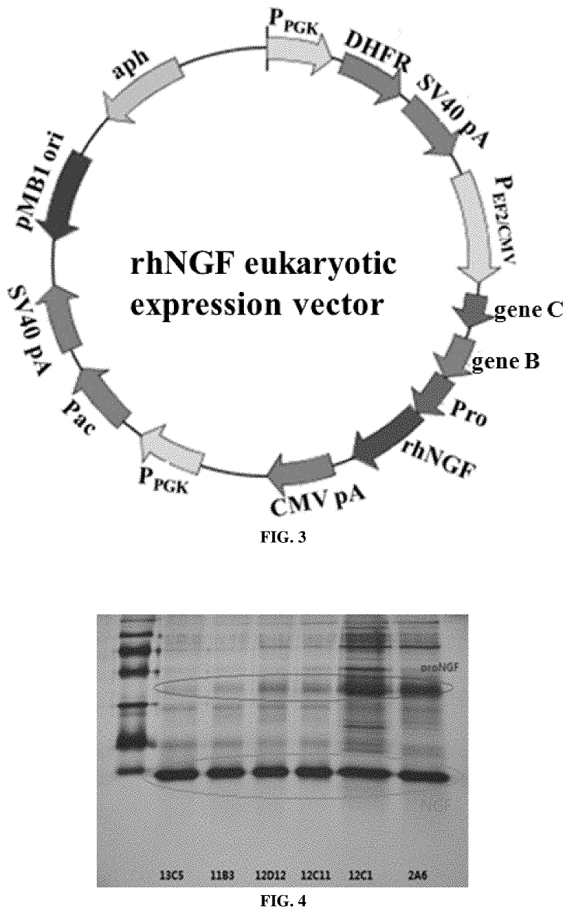

The linearized expression vectors p-glo-Pre-pro-rhNGF(SfiI) and p-aden-Pre-pro-rhNGF(SfiI) were transiently transfected into the CHO cells, cultured for 48 hours, and then sampled. Expression efficiency was assessed by detecting the rhNGF contents of the supernatants with ELISA, and each independent experiment wasonducted twice as shown in FIG. 2. The experiment results show that adding an intron to the 5′ end of a signal peptide-proNGF gene combination was indeed capable of increasing the expression level of rhNGF significantly, and that there was no marked difference between the two introns. It is thus confirmed that an intron-signal peptide-proNGF gene combination is a better choice for rhNGF expression. As shown in FIG. 3, the genetic elements of each gene combination inserted into the corresponding eukaryotic expression vector were an intron, a signal peptide, and the rhNGF gene, in that order.

### Embodiment 2: Establishment of an Engineered Cell Strain

1. The CHO Cell Culture Conditions and the Cell Transfection Method were the Same as in 3.3.

### 2. Stability Screening

48 hours after transfection, the cells were divided into two parts. One part was added with 10 μg/mL puromycin and 100 nM MTX, and the other part with 20 μg/mL puromycin and 200 nM MTX. Once cell viability was restored to 85% or above, each part of cells was further divided into two parts, one of which was added with 30 μg/mL puromycin and 500 nM MTX, and the other of which was added with 50 μg/mL puromycin and 1000 nM MTX. The screening process continued, and the criterion for terminating the screening process was that cell viability exceeded 90%. A total of six cell pools were obtained after two rounds of screening, and their specific yields were analyzed. Cell pools with high specific yields and good cell growth were selected for monocloning.

### 3. Monocloning by the Limiting Dilution Method and Clone Screening

The clone culture medium was FortiCHO added with 6 mM glutamine. The to-be-cloned cells were diluted to 2˜5 cells/mL. Using an 8-channel pipette, the cell suspension was added to a 96-well plate at 200 μL per well. The cells were placed into a carbon dioxide incubator and were incubated at 37° C. in 5% carbon dioxide. After incubation for 11˜14 days (depending on the cloning speed), 20 μL was sampled from each well where monoclones had formed, and the rhNGF concentrations of the samples were analyzed by ELISA. Clones with high expression levels were transferred from the 96-well plate to a 48-well plate and were added with 200 μL of fresh culture medium, followed by the screening reagents MTX and puromycin until the pre-monocloning cell pool screening concentration was reached. When the degree of confluence arrived at 100%, subculturing in a 12-well plate was performed. Once the cells in the 12-well plate reached the subculturing density, they were transferred into a centrifuge tube and centrifuged. After removing the supernatant, the cells were washed once with PBS, re-suspended in 1 mL of fresh culture medium, and then added to a 6-well plate. 30 μL of the cell suspension was taken out for an analysis of cell density. The 6-well plate was then put into the incubator for 2˜4-hour incubation. After that, 100 μL of the culture solution was taken out and centrifuged in order to obtain the supernatant. The incubation continued after each well of the 6-well plate was added with 1 mL of fresh culture medium and the screening reagents. The rhNGF concentration of the culture supernatant was analyzed by ELISA. The specific yield of cells was calculated with the following equation: specific yield=rhNGF concentration/cell density/incubation time. Based on their specific yields, the clones were screened for a second time.

The cells obtained by screening were subjected to a subculture stability test. Six cell strains that performed well in the stability test were batch-cultured, and the rhNGF in the supernatants of the batch cultures were preliminarily purified with a Capto S chromatography column and then analyzed by SDS-PAGE. The SDS-PAGE analysis results in FIG. 4 show that the rhNGF expressed by the batch-cultured 13C5 cell had a relatively low proNGF protein content. As the proNGF protein is a product-related impurity, the lower the proNGF protein content the better. The 13C5 cell was therefore chosen as the engineered cell strain to be used.

Embodiment 3: Determination of the Growth Curve, Cell Viability, and rhNGF Expression Level Variation Trend of the Engineered Cell Strain in a Bioreactor

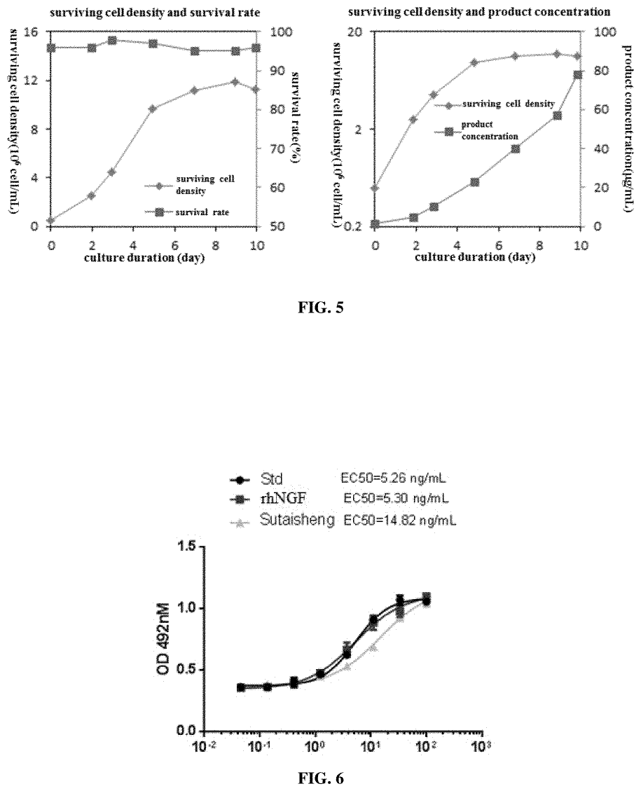

The engineered cell strain was produced and cultured by the fed-batch culture method. The culture scale was increased from a 30-mL shake flask to a 2.5-L one and then to a 28-L mechanical-agitation bioreactor capable of sanitization in place, wherein the bioreactor was equipped with a single inclined agitator blade, had an agitator blade rotation speed of 125 rpm during operation, and featured large-bubble aeration. The control of dissolved oxygen began with a cascade control of the air flow rate. When the gas flow rate reached the highest setting, oxygen was introduced, and the air flow rate was reduced at the same time such that the total flow rate remained unchanged. The pH value was kept at 7.2 in the initial stage of the culture process by controlling the CO₂ flow rate. As cells grew, the pH value was lowered and then bounced back. Once the pH value reached 7.2 (the preset value), dilute hydrochloric acid was used to control the pH value at the preset value until the culture process ended. The cells were sampled on a regular basis during the culture in order to monitor cell density, cell viability, and the concentration of rhNGF. The monitoring results are summarized in FIG. 5.

According to the monitoring results, the fifth day of the culture saw the growth of cells enter the plateau phase from the exponential phase, the density of living cells was generally stable from the sixth to the tenth day, with the highest cell density being 1.2×10⁷/mL, and cell viability stayed higher than 90%. The rhNGF concentration kept increasing at high speed during the culture process and arrived at 78 mg/L at the end of the process.

Embodiment 4: Determination of the Biological Activity of rhNGF by TF-1 Cell/MTS Colorimetry

Recorded in Volume III of the 2015 edition of the Pharmacopoeia of the People's Republic of China, TF-1 cell/MTS colorimetry is a classical method for assaying the biological activity of rhNGF. The biological activity of the rhNGF was assayed by this method and was compared with those of the international standard (Product No. 93/556, NIBSC) and of an mNGF for injection use (Product Name: Sutaisheng, Staidson (Beijing) Biopharmaceuticals Co., Ltd.).

Well grown TF-1 cells (cells derived from the human erythroleukemia) of the adapted, NGF-dependent type (obtained from the Recombinant Protein Laboratory of the National Institutes for Food and Drug Control of the People's Republic of China) were inoculated into a 96-well plate at 5000 cells per well along with a basic culture medium (1640+10% FBS+1% P/S), with the volume of each well being 100 μL. Then, each well was added with 100 μL of a to-be-tested NGF (the rhNGF, the international standard (Std), or Sutaisheng) solution, whose NGF concentration was set at 100, 33, 11, 3.3, 1.1, 0.33, 0.11, and 0.033 ng/mL, with each concentration used in duplicate wells. Once the mixture in each well was thoroughly mixed, the plate was placed in a 37° C., 5% CO₂ incubator for 72-hour incubation. After that, each well was added and thoroughly mixed with 20 μL of MTS, and incubation continued at 37° C. for another 3 hours. The OD value of each well was then measured with an ELISA instrument at 492 nm, and each set of data was fitted with an absorbance-concentration curve by using the software Grandpad 6.0 (a four-parameter nonlinear regression equation was selected to fit the data). In addition, the EC₅₀ value of each sample with regard to the induction of TF-1 cell proliferation was calculated. The measurement, curve fitting, and calculation results are shown in FIG. 6.

According to the measurement, curve fitting, and calculation results, the activity of the rhNGF in inducing TF-1 cell proliferation was equivalent to that of the international standard (Std) (the EC₅₀ values being 5.30 ng/mL and 5.26 ng/mL respectively) and was higher than that of Sutaisheng (whose EC₅₀ values was 14.82 ng/mL).

What is claimed is:

1. A gene combination for expressing recombinant human nerve growth factor (rhNGF), comprising:
an rhNGF precursor gene, and an intron, wherein said intron comprises the nucleotide sequence shown in SEQ ID NO: 1 or SEQ ID NO: 2.

2. The gene combination of claim 1, wherein said rhNGF precursor gene has the nucleotide sequence shown in SEQ ID NO. 7.

3. A eukaryotic expression vector for rHNGF expression comprising the gene combination of claim 2.

4. The eukaryotic expression vector for rHNGF expression of claim 3, further comprising a nucleotide sequence encoding a signal peptide.

5. The eukaryotic expression vector for rHNGF expression of claim 4, wherein the nucleotide sequence comprises the sequence of SEQ ID NO:3 or SEQ ID NO:5.

6. A CHO cell comprising a eukaryotic expression vector of claim 3.

7. A method of preparing rhNGF comprising expressing rHNGF by a CHO cell transfected which a eukaryotic expression vector of claim 3.

8. A method of preparing rhNGF comprising using an intron comprising the nucleotide sequence shown in SEQ ID NO. 1 or SEQ ID NO. 2.


SEQUENCE LISTING (10 sequences)

SEQ ID NO: 1 - DNA, 150 bp, Artificial Sequence
  Synthetic
ctcgactgatcacaggtaagtatcaaggttacaagacaggtttaaggagaccaatagaaactgggcttgtcgagacagagaagactcttgcgtttctgataggcacctattggtcttactgacatccactttgcctttctctccacaggt

SEQ ID NO: 2 - DNA, 296 bp, Artificial Sequence
  Synthetic
gaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcg

SEQ ID NO: 3 - DNA, 90 bp, Artificial Sequence
  Synthetic
atggactctaaaggctcctctcagaagggtagtaggctgctgctgctgctggtggtgtcaaatctgctgctgtgccagggggtcgtcagc

SEQ ID NO: 4 - protein, 30 aa, Bos taurus
MDSKGSSQKGSRLLLLLVVSNLLLCQGVVS

SEQ ID NO: 5 - DNA, 51 bp, Artificial Sequence
  Synthetic
atgggtgtcaaggtcctgttcgcactgatttgtatcgccgtcgcagaagcc

SEQ ID NO: 6 - protein, 17 aa, gaussia
MGVKVLFALICIAVAEA

SEQ ID NO: 7 - DNA, 663 bp, Artificial Sequence
  Synthetic
gagcctcatagtgaatcaaacgtgcctgctggccacaccatcccacaggcacattggacaaagctgcagcacagcctggacacagctctgaggcgggcacgctctgccccagccgctgcaatcgccgctcgcgtcgccggacagactcgaaatattaccgtggaccccaggctgttcaagaaaagacgcctgcgatcacctcgtgtcctgttttccactcagccccctcgagaggcagccgatacccaggacctggatttcgaagtgggcggagctgcacccttcaacaggacccaccggagtaagagatccagctctcaccccatcttccatcggggggagttctccgtgtgcgattccgtgagcgtctgggtgggtgacaaaaccacagctacagatatcaagggcaaagaggtcatggtgctgggagaagtcaatattaacaattccgtgttcaagcagtacttctttgaaactaaatgccgtgacccaaaccccgtcgattccgggtgtagaggtattgactctaagcattggaatagttattgtactaccacacacacatttgtgaaggccctgactatggatggcaaacaggccgcttggagattcattcgtattgacactgcttgcgtctgcgtgctgagtcgtaaggctgtgcgg

SEQ ID NO: 8 - protein, 221 aa, Homo sapiens
EPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVR

SEQ ID NO: 9 - DNA, 54 bp, Artificial Sequence
  Synthetic
atgtcaatgctgttttacactctgattaccgcttttctgatcggaatccaggcc

SEQ ID NO: 10 - protein, 18 aa, Homo sapiens
MSMLFYTLITAFLIGIQA